(19) United States
(12) Patent Application Publication
(10) Pub. No.: US 2020/0116874 A1
(43) Pub. Date: Apr. 16, 2020

(54) X-RAY PULSING DURING SENSOR OPERATION FOR HIGH FLUX PHOTON COUNTING COMPUTED TOMOGRAPHY (CT) IMAGING SYSTEM APPLICATIONS

(71) Applicant: REDLEN TECHNOLOGIES, INC., Saanichton (CA)

(72) Inventors: Georgios PREKAS, Victoria (CA); Saeid TAHERION, Victoria (CA)

(21) Appl. No.: 16/199,588

(22) Filed: Nov. 26, 2018

Related U.S. Application Data
(60) Provisional application No. 62/744,289, filed on Oct. 11, 2018.

(51) Int. Cl. G01T 1/17; A61B 6/03; G21K 1/04

(52) U.S. Cl. CPC G01T 1/17; G01T 1/29; G21K 1/04; A61B 6/032

(57) ABSTRACT

Various embodiments include an imaging system and methods of operating the system to reduce effects from space charge formation in radiation detectors. The imaging system includes a radiation detector configured to detect photon energy from ionizing radiation, a source of ionizing radiation configured to emit a beam of radiation toward the radiation detector, and a chopper disposed between the radiation detector and the source of ionizing radiation, wherein the chopper is configured to periodically block the beam of radiation from reaching the radiation detector. The chopper may be configured to limit delivery of photon energy to the radiation detector to durations shorter than an onset time of dynamic polarization and E-field relaxation. In some embodiments, the chopper is a rotating chopper rotated by a drive motor. In some embodiments, the chopper is a shutter.

### RELATED APPLICATIONS

The present application claims the benefit of priority to U.S. Provisional Application No. 62/744,289 entitled “Method of X-ray and Bias Pulsing During CdZnTe Sensor Operation for High Flux Photon Counting CT (PCCT) Applications,” filed Oct. 11, 2018, the entire contents of which are hereby incorporated by reference.

### FIELD

The present application relates generally to imaging devices that employ radiation detectors for photon counting, and specifically to a method and an apparatus for operating cadmium zinc telluride (CdZnTe) sensors under high flux conditions.

### BACKGROUND

In computed tomography (CT) imaging systems, an X-ray source emits a fan-shaped beam toward an object, which may be, for example, a piece of baggage at an airport scanner or a patient in a medical diagnostic clinic, or any other biological or non-biological object under imaging. The X-ray beam is attenuated by the object, and is subsequently detected by a detector array, such as a CdZnTe detector. Other direct conversion detectors employing cadmium telluride (CdTe), gallium arsenide (GaAs), or silicon (Si), or any indirect director based on a scintillator material, may also be used in CT imaging systems. Image slices collected by scanning the object can, when joined together, reconstruct 3-dimensional cross-section images of the object.

In typical CT imaging systems, an array of radiation detectors includes a number of solid-state detector elements (which may be arranged as pixels for imaging) that each produce a dedicated electrical signal indicating an amount of radiation reaching the detector element. The electrical signals may be transmitted to a data processing card for analysis. Finally, using image reconstruction techniques, a reconstruction image may be produced. The intensity of the attenuated beam received by each detector element depends upon the attenuation of the X-ray beam by the object. For example, when scanning a human body, bone turns up white, air turns up black, and tissues and mucous turn up in shades of gray.

### SUMMARY

Various embodiments of the present disclosure suppress temporal dynamic charge build up and polarization, which otherwise occurs during contemporary X-ray sensor operation, in order to achieve improved dynamic responses and stability for high flux photon counting applications. In particular, various embodiments include a chopper interposed between the radiation detector and the source of ionizing radiation to interrupt projected beams of radiation at a very high frequency during active data acquisition by the radiation detector. The chopper may be operated at high enough rates to provide a pulsed radiation delivery technique with pulse durations shorter than the onset time of dynamic polarization and E-field relaxation within the radiation detector.

Various embodiments of the present disclosure include an imaging device comprising a radiation detector configured to detect photon energy from ionizing radiation, a source of ionizing radiation configured to emit a beam of radiation toward the radiation detector, and a chopper disposed between the radiation detector and the source of ionizing radiation, wherein the chopper is configured to periodically block the beam of radiation from reaching the radiation detector. In various embodiments of the imaging device, the chopper is configured to limit delivery of photon energy to the radiation detector to durations shorter than an onset time of dynamic polarization and E-field relaxation. In some embodiments of the imaging device, the chopper is a rotating chopper. In some embodiments of the imaging device, the chopper is a pneumatic shutter. In some embodiments of the imaging device, the chopper is formed from a material selected from the group tungsten, lead, or terbium. In some embodiments of the imaging device, the ionizing radiation is X-ray radiation. In some embodiments of the imaging device, the ionizing radiation is gamma radiation. In some embodiments of the imaging device, the source of ionizing radiation is configured to emit the beam of radiation at an adjustable delivery rate. In some embodiments of the imaging device, the chopper is integrated into the source of ionizing radiation such that the source of ionizing radiation is configured to emit radiation in a series of pulses. In some embodiments of the imaging device, the radiation detector comprises cadmium zinc telluride (CdZnTe). In some embodiments of the imaging device, the radiation detector is configured to alternate between on-periods of data acquisition of detected photon energy and off-periods in which no data is acquired from the radiation detector. Some embodiments of the imaging device further comprise a bias power supply configured to selectively apply a voltage to the radiation detector and alternate between on-periods in which an operating voltage is applied to the radiation detector and off-periods in which no voltage is applied to the radiation detector.

Some embodiments of the present disclosure include a method of imaging an object using ionizing radiation, comprising positioning a chopper between a source of ionizing radiation configured to emit a beam of radiation and a radiation detector configured to detect photon energy from ionizing radiation, positioning the object between the chopper and the radiation detector, and activating the chopper to periodically block the beam of radiation from reaching the radiation detector while the source of ionizing radiation is activated. Some embodiment methods further comprise acquiring data from the radiation detector by a computing device, generating an image by the computing device based on the acquired data, determining by the computing device whether the generated image includes an artifact, and adjusting a speed of the chopper by the computing device in response to determining that the generated image includes an artifact.

Some embodiments of the present disclosure include a chopper for use in an imaging system, comprising a radiation-absorbing material configured to periodically block radiation from a radiation source and periodically permit radiation from the radiation source to pass. In some embodiments the chopper comprises a rotatable disk of the radiation-absorbing material configured with a plurality of spaced apart openings, and a drive motor coupled to the rotatable disk of the radiation-absorbing material and configured to rotate the disk during operation of the imaging system. In some embodiments the chopper comprises a plurality of spaced apart blades of the radiation-absorbing material coupled to a hub, and a drive motor coupled to the hub and configured to rotate the hub during operation of the imaging system. In some embodiments the chopper comprises a plurality of spaced apart shield blocks of the radiation-absorbing material coupled to a hub, and a drive motor coupled to the hub and configured to rotate the hub during operation of the imaging system. In some embodiments the chopper comprises a shutter.

### BRIEF DESCRIPTION OF THE DRAWINGS

The accompanying drawings are presented to aid in the description of embodiments of the disclosure and are provided solely for illustration of the embodiments and not limitation thereof.

FIG. 1 is a block diagram of an X-ray imaging system suitable for use with various embodiments of the present disclosure.

FIGS. 2A-2F are drawings of alternative configurations for a rotating chopper according to various embodiments of the present disclosure.

FIG. 3A is a conceptual diagram of a semiconductor radiation detector illustrating X-ray interactions generating electron-hole pairs.

FIG. 3B is a conceptual diagram of a semiconductor radiation detector illustrating how a high X-ray flux can cause a space charge to develop within the detector materials.

FIG. 4A is a graph of a timing sequence of operation of the imaging device in accordance with various embodiments.

FIG. 4B is a relief view of FIG. 4A at 450, in accordance with various embodiments.

FIG. 5 is a graph illustrating output counts of a conventional semiconductor radiation detector.

FIG. 6 is a graph illustrating output counts of a radiation detector used with a chopper in accordance with various embodiments.

FIG. 7 is a process flow diagram illustrating a method of improving the performance of an X-ray imaging system according to embodiments of the present disclosure.

### DETAILED DESCRIPTION

Various embodiments use a high frequency apparatus which may create “pulsed” X-rays at a very high rate (i.e., a pulsed X-ray mode), while operating a radiation detector for high flux spectral photon counting computerized tomography (PCCT). The radiation detector core material (e.g., CdZnTe) may also be biased by a direct current or in a pulsed bias mode. By causing X-rays to be pulsed at a pulse duration of a between a few tens of a microsecond to milliseconds, faster than that compared to the rate of the onset of dynamic polarization and E-field relaxation, the space charge formation caused by charge carrier trapping may be significantly suppressed. Combining this pulsed X-rays mode with bias power supply pulsing at a high rate may allow recombination of space charges, thus retaining a uniform E-field that is not influenced by memory (relaxation) from previous X-ray exposures. Biasing the X-ray detector may encourage additional trapping by enhancing recombination between the charge carriers. Various embodiments aim to deliver packets of X-ray photons at a very fast rate, to balance the charge carrier trapping time, trap residence time and charge carrier de-trapping time, thus achieving low density space charge which could be rapidly cleared by simultaneously switching the X-ray and the bias.

The electrical signal generated by solid state radiation detectors, such as CdZnTe detectors, in CT and similar imaging systems results from X-rays exciting electrons in the atoms of the material that ejects electrons from their orbits and into a conduction band of the bulk material. Each electron ejected into the conduction band leaves behind a net positive charge that behaves like a positively charged particle known as a “hole” that migrates through the material in response to an electric field applied between a cathode and an anode. Electrons in the conduction band are attracted by the resulting internal electric field and migrate to the anode where they are collected creating a small current that is detected by circuitry, while the holes migrate towards the cathode. Each X-ray or gamma-ray will generate many electron-hole pairs, depending upon the energy of the photons. Electrons migrate quickly to the anode, while holes migrate slower and may be trapped by defects.

A semiconductor radiation detector typically includes defects (e.g., dopants, vacancies, lattice defects, etc.) that can trap charge carriers (e.g., holes and/or electrons), and thus slow the migration of charges to the anode and cathode. Referred to as deep level defects, trapping charge carriers can result in the buildup of a space-charge that affects the internal electric field that may cause dynamic effects and/or reduce the efficiency of the detector. Additionally, holes in a semiconductor exhibit an effective mass depending upon which electron was ejected to create the hole. Holes with higher effective mass drift slower towards the cathode that lighter holes and electrons move faster than holes towards the anode.

Also, as a result of deep traps and the slower migration of holes, when a radiation detector is subject to a high X-ray flux, a positive space charge can form in the detector. This positive space charge may reduce the internal electrical field in the detector, which may degrade performance of the detector. The effects of deep traps and the build up of a space-charge within a detector are temporally dynamic, resulting in changes in the efficiency of the detector as a function of time from the start of radiation.

Various embodiments provide methods and structures to suppress the temporal dynamic charge build up and polarization that traditionally occur during sensor operation (due to X-ray or dark current), and thereby provide better dynamic response and stability in detector efficiency and output, particularly for high flux photon counting applications. Various embodiments reduce the amount of the space charge in the CdZnTe while readings are taken by periodically preventing X-rays from reaching the detector thereby providing alternating durations of irradiation and no-irradiation in accordance with the E-field relaxation times (i.e., trapping and de-trapping rates). In an embodiment, a radiation chopper or shutter (as described below) is positioned in an imaging system (e.g., a CT imaging system) to periodically block X-rays emitted by the radiation source from reaching the detector. The duration of X-ray exposure in each period of exposure (referred to herein as “X-ray exposure period”) and/or duration when X-rays are blocked may vary from tens of microseconds to hundreds of milliseconds depending on the X-ray flux and the amount of space charge formation exhibited in the detector. For example, the X-ray exposure periods may be much shorter than the data acquisition periods (i.e., the time that data from the detector(s) is collected) during an imaging session. X-ray exposure periods from less than lms to tens of microseconds may be achieved by adjusting the rate of revolution of a chopper or the open-close position frequency of a pneumatic shutter and yet maintaining high counting statistics. Breaking up an X-ray imaging session into a sequence of brief exposures interspersed with periods of no exposure may suppress temporal dynamic charge build up and polarization in solid state radiation detectors, thereby improving the dynamic responses and stability for high flux photon counting imaging applications. Various embodiments improve the dynamic response and stability of CdZnTe radiation detectors/sensors under intense and rapidly changing X-ray irradiation environments, such as in a medical Photon Counting CT scanner. Various embodiments may also be implemented in other applications in which solid state radiation detectors operate in conditions of rapidly changing (microsecond range) X-ray intensity (i.e., flux) and/or energy, such as security baggage scanners and non-destructive imaging/testing.

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the claims. Any reference to claim elements in the singular, for example, using the articles “a,” “an,” or “the” is not to be construed as limiting the element to the singular. The terms “example,” “exemplary,” “e.g.,” or any term of the like are used herein to mean serving as an example, instance, or illustration. Any implementation described herein as an “example” is not necessarily to be construed as preferred or advantageous over another implementation. The drawings are not drawn to scale. Multiple instances of an element may be duplicated where a single instance of the element is illustrated, unless absence of duplication of elements is expressly described or clearly indicated otherwise.

As used herein, the expression “ionizing radiation” refers to radiation consisting of particles, X-rays, or gamma rays with sufficient energy to cause ionization in the medium through which it passes. For ease of explanation herein, X-rays are referred to as the type of ionizing radiation used. However, this short hand reference is not intended to limit the claims to just X-ray applications unless specifically recited in the claims.

As used herein, the term “chopper” refers to a device in the form of a rapidly moveable radiation shield that interrupts a beam of radiation at short regular intervals. The chopper may be positioned within an imaging system, such as a computed tomography (CT) imaging system, so as to periodically block X-rays from the radiation source from reaching the radiation detector during active data acquisition. A chopper may be used with any form of ionizing radiation, but is particularly useful for imaging systems having a high flux, such as a medical X-ray CT imaging system or a gamma ray luggage scanning system.

References to “detectors” and “radiation detectors” encompass any form of solid state radiation detectors, such as semi-conductor type radiation detectors. In a particular embodiment, radiation detectors may be CdZnTe radiation detectors/sensors, including such detectors in the form of an array of pixel detectors suitable for imaging in CT imaging systems.

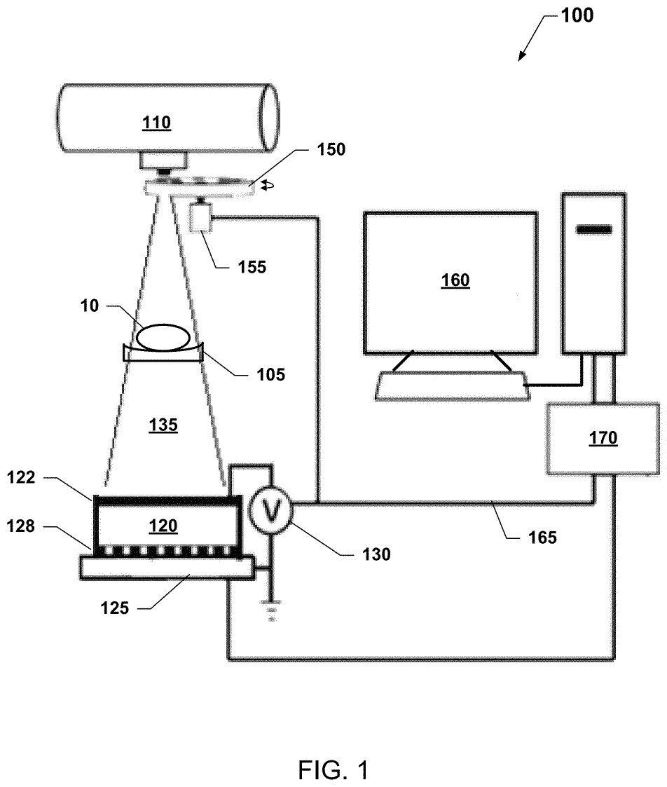

FIG. 1 is a functional block diagram of a CT imaging system 100 in accordance with various embodiments. The CT imaging system 100 may include an X-ray source 110 (i.e., a source of ionizing radiation), a radiation detector 120, and a chopper 150 disposed between the radiation detector 120 and the X-ray source 110. The CT imaging system 100 may additionally include a support structure 105, such as a table or frame, which may rest on the floor and may support an object 10 to be scanned. The support structure 105 may be stationary (i.e., non-moving) or may be configured to move relative to other elements of the CT imaging system 100. The object 10 may be all or a portion of any biological (e.g., human patient) or non-biological (e.g., luggage) object to be scanned.

The X-ray source 110 is configured to deliver ionizing radiation to the radiation detector 120 by emitting an X-ray beam 135 toward the radiation detector 120, which is also toward the object 10 temporarily positioned between the X-ray source 110 and the radiation detector 120. After the X-ray beam 135 is attenuated by the object 10, the beam of radiation 135 is received by the radiation detector 120. The chopper 150 may be positioned closer to the X-ray source 110 than the radiation detector 120, such as immediately adjacent to an X-ray tube aperture of the X-ray source 110. The chopper 150 may be incorporated into or with the X-ray source 110 and configured to provide pulses of X-rays 135 at a high frequency.

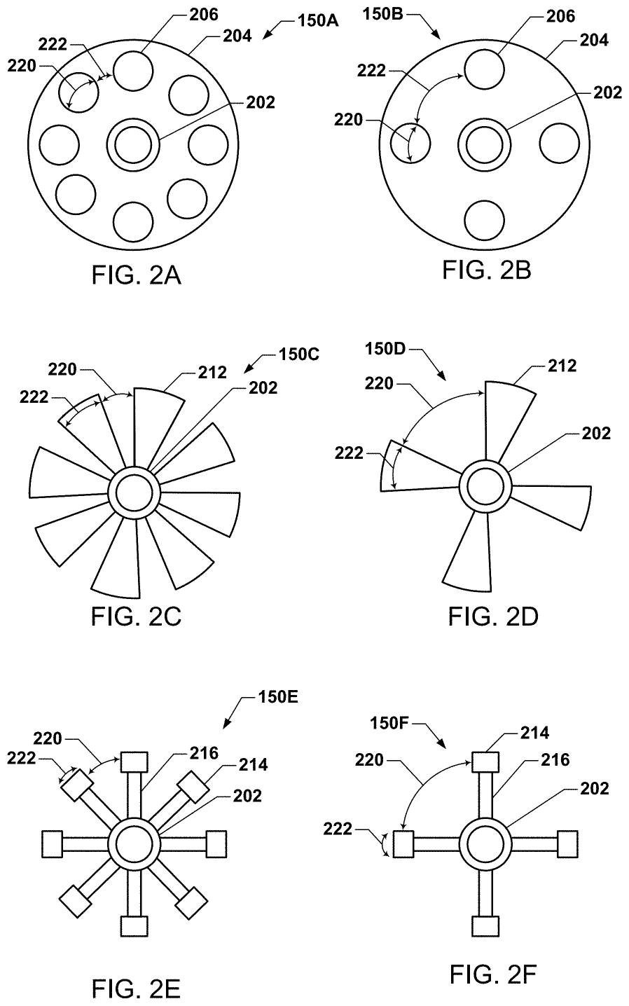

In the embodiment illustrated in FIG. 1, the chopper 150 is a high frequency rotating assembly with apertures or openings spaced apart by radiation absorbing material. In the illustrated embodiment, the chopper 150 is in the form of a disk of material that will attenuate or block ionizing radiation, such as tungsten, lead or steel, configured with spaced apart openings or apertures. However, other configurations are possible, some examples of which are illustrated in FIGS. 2A-2F described below.

When the chopper 150 is rotated, such as by a drive motor 155, the chopper periodically positions radiation-absorbing material in the path of the x-rays to prevent (or attenuate) ionizing radiation from reaching the detector 120 except when the apertures/openings aligned with the X-ray beam 135 (i.e., align with a line between the X-ray source and the detector 120) A rotational axis of the rotational motor 155 may extend parallel to a direct line between the X-ray tube aperture and the radiation detector. During rotation of the chopper 150, radiation 135 is allowed to reach the radiation detector 120 during the duration that each aperture/opening in the chopper assembly aligns with the source of radiation 110, and radiation 135 is blocked (or attenuated) from reaching the radiation detector 120 by the radiation-absorbing material at other times. By adjusting the size of apertures/openings, span of the radiation absorbing material between apertures/openings and the rotation rate of the chopper, the duration of detector exposures and the duration between exposures can be controlled. Spinning a rotating chopper 150 at high speed permits exposure and non-exposure durations to be in the millisecond range.

In an alternative embodiment, the chopper 150 may be a shutter made of radiation blocking material (e.g., W) that is capable of opening and closing at a high frequency, such as a high frequency pneumatic shutter, that may be positioned in, at or near an aperture of the radiation source.

The X-ray source 110 and radiation detector 120 may be stationary or incorporated into a moving assembly, such as a rotating gantry, for 360-degree imaging of the object 10. Similarly, although the chopper 150 may be configured to rotate at high frequencies, a support structure of the chopper 150 (e.g., the high frequency rotational motor 155) may be stationary at least with respect to the X-ray source 110 and/or the radiation detector 120.

The radiation detector 120 may be controlled by a high voltage bias power supply 130 that selectively creates an electric field between an anode 122 and cathode 128 pair coupled thereto. The radiation detector 120 may include CdZnTe material disposed between the anode 122 and cathode 128 and thus configured to be exposed to the electrical field therebetween. A read-out application specific integrated circuit (ASIC) 125 coupled to the anode 122 and cathode 128 pair may receive signals (e.g., charge or current) from the anode 122 and be configured to provide data to and by controlled by a control unit 170.

The control unit 170 may be configured to synchronize the X-ray source 110, the read-out ASIC 125, the high voltage bias power supply 130, and the chopper 150 via the rotational motor 155. The control unit 170 may be coupled to and operated from a computing device 160. Alternatively, the computing device 160 and the control unit 170 may be integrated together as one device.

The object 10 may slowly pass between the X-ray source 110 and the radiation detector 120 or alternatively the object may remain stationary while the X-ray source 110 and the radiation detector 120 move relative to the object 10. Either way, the radiation detector 120 may capture incremental cross-sectional profiles of the object 10. The data acquired by the radiation detector 120 may be passed along to the computing device 160 that may be located remotely from the radiation detector 120 via a connection 165. The connection 165 may be any type of wired or wireless connection. If the connection 165 is a wired connection, the connection 165 may include a slip ring electrical connection between any structure supporting the radiation detector 120 and a stationary support part of the support structure 105, which supports any part (e.g., a rotating ring). If the connection 165 is a wireless connection, the radiation detector 120 may contain any suitable wireless transceiver to communicate data with another wireless transceiver that is in communication with the computing device 160. The computing device 160 may include processing and imaging applications that analyze each profile obtained by the radiation detector 120, and a full set of profiles may be compiled to form two-dimensional images of cross-sectional slices of the object 10.

Various alternatives to the design of the CT imaging system 100 of FIG. 1 may be employed to practice embodiments of the present disclosure. CT imaging systems may be designed in various architectures and configurations. For example, a CT imaging system may have a helical architecture. In a helical CT imaging scanner, the X-ray source and detector array are attached to a freely rotating gantry. During a scan, a table moves the object smoothly through the scanner creating helical path traced out by the X-ray beam. Slip rings enable the transfer of power and data on and off the rotating gantry. In other embodiments, the CT imaging system may be a tomosynthesis CT imaging system. In a tomosynthesis CT scanner, the gantry may move in a limited rotation angle (e.g., between 15 degrees and 60 degrees) in order to detect a cross-sectional slice of the object. The tomosynthesis CT scanner may be able to acquire slices at different depths and with different thicknesses that may be constructed via image processing.

The detector array of a CT imaging system may include an array of radiation detector elements, such as pixel sensors. The signals from the pixel sensors may be processed by a pixel detector circuit, which may sort detected photons into energy bins based on the energy of each photon or the voltage generated by the received photon. When a photon is detected, its energy is determined and the photon count for its associated energy bin is incremented. For example, if the detected energy of a photon is 24 kilo-electron-volts (keV), the photon count for the energy bin of 20-40 keV may be incremented. The number of energy bins may range from one to several, such as two to six. In an illustrative example, a photon counting detector may have four energy bins: a first bin for detecting photons having an energy between 20 keV and 40 keV, a second bin for detecting photons having an energy between 40 keV and 60 keV, a third bin for detecting photons having an energy between 60 keV and 80 keV, and a fourth bin for detecting photons having an energy above 80 keV. The greater the total number of energy bins, the better the material discrimination.

In CT imaging systems, a scanned object is exposed to an X-ray beam and attenuated photons from the X-ray beam are detected and counted by individual radiation detector pixels in a detector array. When an object (e.g., the object 10) is loaded in a CT imaging system, the X-ray beam may be heavily attenuated, and the number of photons detected by the detector array may be orders of magnitude less than the number of photons emitted from an X-ray source. For image reconstruction purposes, the radiation detector can be exposed to a direct X-ray beam without an intervening object located inside the CT imaging system. In such cases, the photon count rates in the CT imaging system may reach values of 100 million counts per second per square millimeter (Mcps/mm²) or more. The detector array may be capable of detecting such a wide range of photon count rates.

FIGS. 2A-2F illustrate different example configurations of a rotating chopper according to various embodiments. Referring to FIGS. 2A-2F collectively, the chopper 150A-150F may include a hub 202 configured to be connected to the drive motor 155 and secure the structures that will block x-rays when the chopper is rotated. The radiation-absorbing material used to block x-rays, may be configured in a variety of different configurations as illustrated.

Referring to FIGS. 2A and 2B, in one configuration, a chopper 150A, 150B maybe in the form of a disk 204 of radiation-absorbing material that includes a number of circumferentially distributed apertures 206 through which x-rays will pass when each aperture is aligned with the x-ray beam 135. The duration of each pulse of x-rays will be a function of the span of the aperture 220 (i.e., diameter of the opening) and the rotation rate of the disk 204. Similarly, the duration that x-rays will be blocked from reaching the detector between each x-ray pulse will be a function of the span of the distance between apertures 222 and the rotation rate of the disk 204. Thus, by adjusting the diameter of each aperture 206 and the spacing of the apertures around the disk 204, as well as controlling the speed of rotation of the chopper 150A, 150B via the drive motor 155, the periods of x-ray exposure and intervening periods of no x-ray exposure can be controlled. Additionally as illustrated in FIG. 2B, the ratio of the duration of irradiation to the duration of no irradiation may be adjusted by changing the number of apertures 206 within the disk 204. In the chopper 150B illustrated in FIG. 2B, there are only four apertures 206, and thus the span 222 or the distance between apertures 206 is larger than the diameter 220 of the apertures, resulting in longer periods without irradiation and shorter periods of irradiation.

Referring to FIGS. 2C and 2D, in another configuration, a chopper 150C, 150D maybe in the form of a set of blades 212 of radiation-absorbing material that are spaced apart providing openings through which x-rays will pass when each opening is aligned with the x-ray beam 135. The duration of each pulse of x-rays will be a function of the span of the openings 220 (i.e., the distance between blades 212) and the rotation rate of the chopper 150C. Similarly, the duration that x-rays will be blocked from reaching the detector between each x-ray pulse will be a function of the span 222 of the blades 212 and the rotation rate of the chopper 150C. Thus, by adjusting the span 222 of each blade 212 and the spacing between blades, as well as controlling the speed of rotation of the chopper 150C, 150D via the drive motor 155, the periods of x-ray exposure and intervening periods of no x-ray exposure can be controlled. Additionally as illustrated in FIG. 2D, the ratio of the duration of irradiation to the duration of no irradiation may be adjusted by changing the number of blades 212 within the chopper 150C, 150D. In the example illustrated in FIG. 2D, there are only four apertures 206, and thus the span 220 of the distance between blades 212 is larger than the span 222 of the blades 212, resulting in longer periods of irradiation and shorter periods without irradiation.

Referring to FIGS. 2E and 2F, in another configuration, a chopper 150E, 150F maybe in the form of a set of shielding blocks 214 of radiation-absorbing material supported on spokes 216 connected to the hub 202 that are spaced apart providing openings between the shield blocks through which x-rays will pass when each opening is aligned with the x-ray beam 135. The duration of each pulse of x-rays will be a function of the span of the openings 220 (i.e., the distance between shield blocks 214) and the rotation rate of the chopper 150E, 150F. Similarly, the duration that x-rays will be blocked from reaching the detector between each x-ray pulse will be a function of the span 222 of the shield blocks 214 and the rotation rate of the chopper 150E, 150F. Thus, by adjusting the span 222 of each shield block 214 and the spacing between shield blocks (or number of shield blocks), as well as controlling the speed of rotation of the chopper 150E, 150F via the drive motor 155, the periods of x-ray exposure and intervening periods of no x-ray exposure can be controlled. Additionally as illustrated in FIG. 2F, the ratio of the duration of irradiation to the duration of no irradiation may be adjusted by changing the number of shield blocks 214 in the chopper 150F. In the example illustrated in FIG. 2F, there are only four shield blocks 214, and thus the span 220 of the distance between shield blocks 214 is larger than the span 222 of the shield blocks 214, resulting in longer periods of irradiation and shorter periods without irradiation.

Radiation-absorbing material (204, 212, 214) used in various configurations of the chopper 150A-150F may be a metal or metal alloy that exhibits high tolerance to radiation damage and has a high atomic number (high Z) to absorb ionizing radiation, some examples of which include tungsten, lead, and terbium.

FIG. 2 illustrates the interactions of an X-ray 310 or gamma ray with the materials in a radiation detector 120 (e.g., CdZnTe detector material). In particular, the photo-electric effect results in complete absorption of the photon energy 312 and the generation of a cloud of electrons 314 and a corresponding cloud of holes 318 based on the energy of the absorbed photon. The generated electron-hole pairs separate under influence of the electric field applied to the radiation detector 120 between the anode 122 and the cathode 128. The generated electrons 314 drift toward the anode 122, and the generated holes drift towards the cathode 128. The collection of electrons 314 at the anode 122 of the detector results in a current that is proportional to the energy of the absorbed photon (i.e., X-ray 310), thereby enabling both detection of the photon and estimation of the photon's energy. Hole 318 mobility in CdZnTe is very low compared to electrons.

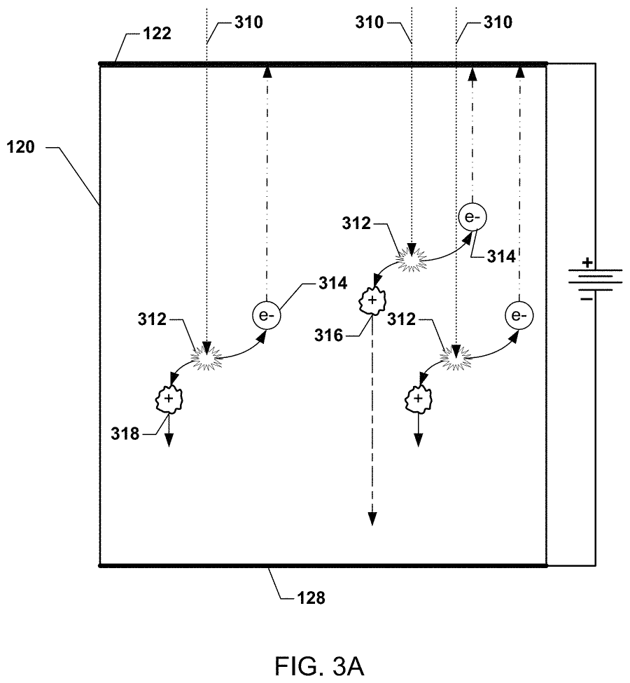

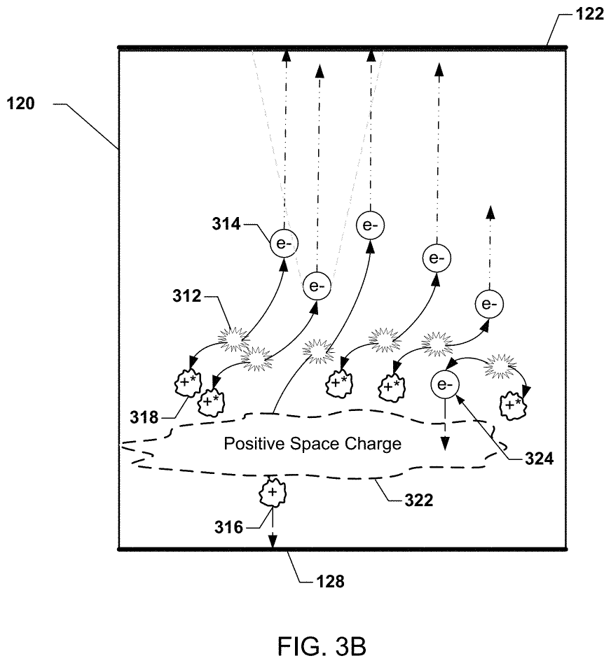

FIG. 3 illustrates how a space charge may form within a radiation detector, such as a CdZnTe radiation detector array, during irradiation due to the formation of positively or negatively stationary charged traps. Depending on the nature of the traps or the impurities and their relative position with respect to the Fermi level in the band gap and their energy, a positive or negative space charge can be formed. This space charge can remain stationary if the conditions that caused the space charge to form do not change.

When such a detector, such as a CdZnTe radiation detector array, is irradiated by relatively high ionizing radiation flux, the formation of a positive space charge 322 may be created mainly by two causes. First, a positive space charge may be created due to ionization of long lifetime deep level hole 318 (i.e., traps). Many impurities (intrinsic or external) can act as trapping sites for holes. Second, a positive space charge may be created due to low or reduced mobility of holes 316 (compared to the mobility of electrons) that are not captured by a trap. In a CdZnTe material the fast-moving electrons are swept away by the electric field, but the slow-moving holes have higher probability of being trapped. Depending on the density of these traps and their characteristics (e.g., life time, energy, cross section and density) and the different X-ray intensities and energies, different numbers of injected electrons and holes will cause different amounts of internal electric field disturbances. Under irradiation of a relatively high flux of ionizing photons, many electrons and hole clouds are formed in the detector by the many X-ray-electron interactions. Due to deep traps and the low mobility of the hole clouds compared to electrons, a positive field charge 322 develops as the holes accumulate in the detector bulk while most electrons 314 are collected by the anode 122. The large positive space charge 322 in the detector reduces the internal electrical field in the detector, impacting the efficiency and responsiveness of the radiation detector. If the internal field is strong enough, some electrons 324 may drift toward the space charge rather than the anode 122, thereby degrading detector performance and accuracy.

In applications in which the X-ray intensity changes rapidly, such as in medical CT imagers or luggage scanners, formation of a space charge due to trapped charge carriers from the injection of holes, changes with time, and the amount of the space charge varies with time. The strength of a space charge may change when the flux of X-ray photons striking the radiation detector material suddenly increases, thus injecting a significant number of electrons and holes that can cause temporal changes in the efficiency of the radiation detector. Thus, initiation of irradiation and changes in the intensity or flux of radiation striking the radiation detector can result in dynamic changes in the internal electric field that drives electrons toward the anode, affecting the spectral and counting output of the radiation detector. This can make the spectral and counting output of the radiation detector time dependent.

A space charge may be formed when operating the radiation detector 120 during biasing (i.e. while emptying of deep traps) by applying a voltage between the anode 122 and cathode 128. Depending on the radiation detector (e.g., CdZnTe material type and contact material), biasing can result in a net negative or net positive space charge forming within the detector material. By creating different domains of electrostatic potential between the anode and the cathode this space charge can dictate the uniformity and/or shape of the internal electric field. Typically, when uniform trapping and space charge formation is assumed between the sensor terminals, a negative space charge will create a linearly changing internal electric field that is higher at the cathode, whereas a positive space charge will create a linearly changing internal electric field that is higher at the anode. This non-uniform internal electric field will influence the transport of electrons, their induction rate at the anode and eventually their signal amplitude of counting pulses.

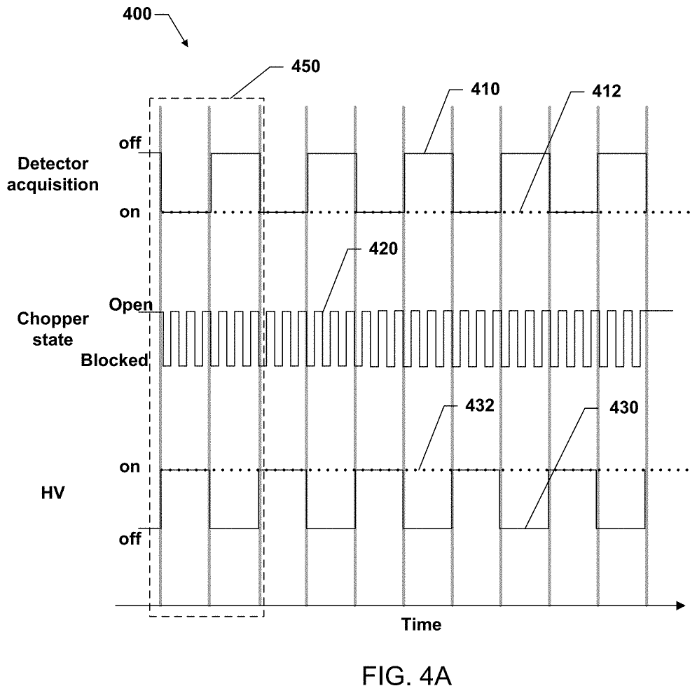

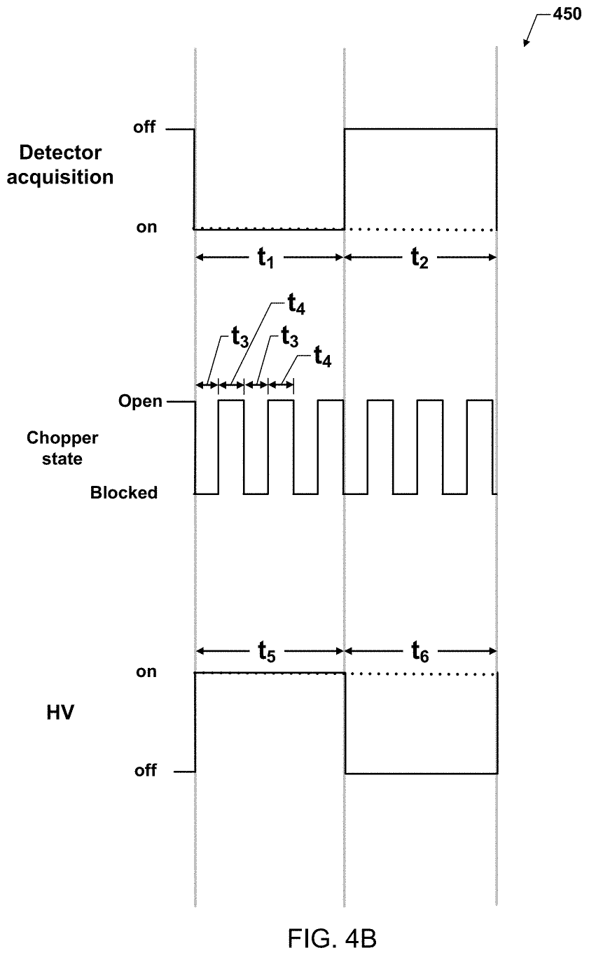

In various embodiments, exposure times used in obtaining images may be coordinated with the x-ray pulses provided by the chopper 150 so that each exposure includes the same number of x-ray pulses, and thus the same amount of energy deposited in the detector. This will help to ensure that images have the same intensity or exposure rate so that image data across a number of images are comparable. FIG. 4A illustrates an example timing sequence of operations of the imaging system 100 of FIG. 1, showing how image durations can be coordinated with x-ray pulses in accordance with various embodiments. An uppermost set of lines 410, 412 represent radiation detector data acquisition (i.e., “Detector acquisition”) when individual images are taken and data is gathered from the radiation detector, and intervals between images (“off” states). The central line 420 represents cycles of the chopper 150 between “open” states when x-rays are permitted to reach the radiation detector, and “blocked” states when x-rays are blocked from reaching the radiation detector. The lowermost set of lines 430, 432 represent high voltage states (i.e., “HV”) within the detector between on states when an x-ray image is obtained and off states between images.

The on-off cycling of the radiation detector (e.g., 120) data acquisition is represented by the alternating detector acquisition line 410 (i.e., the uppermost solid line). The alternating detector acquisition line 410 demonstrates how radiation detector data acquisition may alternate between periods of gathering data to obtain an image (i.e., “on”) during which the radiation detector is energized with a bias voltage (line 430) to detect photons, and off periods during which the radiation detector is not powered on and not outputting data. The data acquisition periods (“on” periods) and the off periods may be the same or different durations. Alternatively, the radiation detector may be powered on continuously (i.e., the bias voltage remains applied even when no image data is gathered), as represented by the continuous HV line 432, but detector data is not recorded between images. As another alternative, the data from the detector may be gathered continuously (i.e., the uppermost dotted straight line 412), but the bias voltage (HV) applied to the detector may be turned off between images (i.e., line 430).

The chopper state line 420 illustrates how the chopper 150 results in periods of irradiation of the detector (i.e., “open” states) interspersed by periods of no irradiation of the detector (i.e., “blocked” states). In various embodiments, the duration of x-ray exposure will be significantly shorter than imaging durations (i.e., detector acquisition and HV on states lines 410, 430), with the duration of radiation exposure and the duration of no irradiation being controlled to achieve a desired level of detector efficiency. As illustrated in FIG. 4A, the periods of data gathering from the detector (i.e. the “on” states in lines 410 and 430) may be synchronized with the chopper so that each imaging duration (i.e. the “on” states in lines 410 and 430) encompasses the same number of periods of irradiation of the detector (i.e., “open” states). This ensures that each image is based on the same amount of x-ray radiation passing through the object being imaged. Without synchronization, the number of periods of irradiation of the detector (i.e., “open” states) per imaging duration may vary.

FIG. 4B is a close-up view of a first timing sequence cycle 450 in FIG. 4A. In various embodiments using alternating detector acquisition (e.g., 410), a data acquisition period t₁ (i.e., Detector acquisition=“on”) and a dead periods t₂ (i.e., Detector acquisition=“off”) may vary from 0.1 ms to 1 ms. In contrast, the duration t₃ of an individual X-ray exposure period provided by a chopper 150 (i.e., Chopper state=“Open”), which is when photons from the emitted X-rays are not blocked by the chopper and reach the X-ray detector, is a substantially shorter period. Similarly, the X-ray recovery period t₄ when X-rays are blocked by the chopper (i.e., Chopper state=“Blocked”)) is also substantially shorter than the data acquisition period t₁. The duration of the X-ray exposure period t₃ and duration of the X-ray recovery period t₄ may be set to improve the performance of the radiation detector. On the other hand, the start and length of each data acquisition period t₁ may be synchronized with the chopper open/close cycle such that the same total number of X-ray exposure periods are encompassed in each data acquisition period t₁. In one embodiment, each data acquisition period t1 may be triggered by or otherwise synchronized to an opening of the chopper (i.e., transition to the chopper open configuration). For example, a controller (e.g., the computer 160) may be configured to receive signal from the drive motor 155 indicating the rotation rate or signaling the start of each full rotation, and coordinate the data acquisition periods to accordingly. As another example, a controller (e.g., the computer 160) may be configured to monitor signals from the radiation detector over a matter of a few seconds to determine the periodicity and duration of X-rays pulses reported by the radiation detector, and use the determined periodicity and duration to synchronize data acquisition periods.

The controller may also control or adjust the duration of data acquisition periods t₁ so that the same number a radiation pulses are received by the radiation detector within each data acquisition period. In the example embodiment illustrated in FIG. 4B, the controller is controlling the start and duration of data acquisition periods t₁ so that a whole number of X-ray exposure periods t₃ and an equal whole number of X-ray recovery periods t₄ added together will equal the data acquisition period t₁. Thus, in this embodiment, the length of the data acquisition period t₁ may be determined based upon a repetition rate N, the duration of the X-ray exposure period t₃ and duration of the X-ray recovery period t₄ as follows:

N×1/(t₃+t₄)=t₁ (1).

According to various embodiments, the repetition rate N may vary from a few tens of microseconds to half a microsecond to be synchronized with the detector acquisition. Similarly, the dead periods t₂ of detector acquisition may be synchronized to coincide with a whole number of X-ray exposure periods t₃ and a whole number of X-ray recovery periods t₄.

Similarly, using an alternating bias power supply (e.g., 430), the HV periods t₅ in which an operating voltage is supplied by the HV bias power supply may be synchronized to coincide with the data acquisition period t₁ and the no voltage periods t₆ in which no power is supplied by the HV bias power supply may be synchronized to coincide with the dead periods t₂ of detector acquisition.

In some embodiments, a small offset period corresponding to a difference between the HV period t₅ and the data acquisition period t₁ may be represented as follows:

Δt=t₅−t₁=t₅−(N×1/(t₃+t₄)) (2).

The small offset period At may be included to ensure that the E-field is already established before the data acquisition time starts.

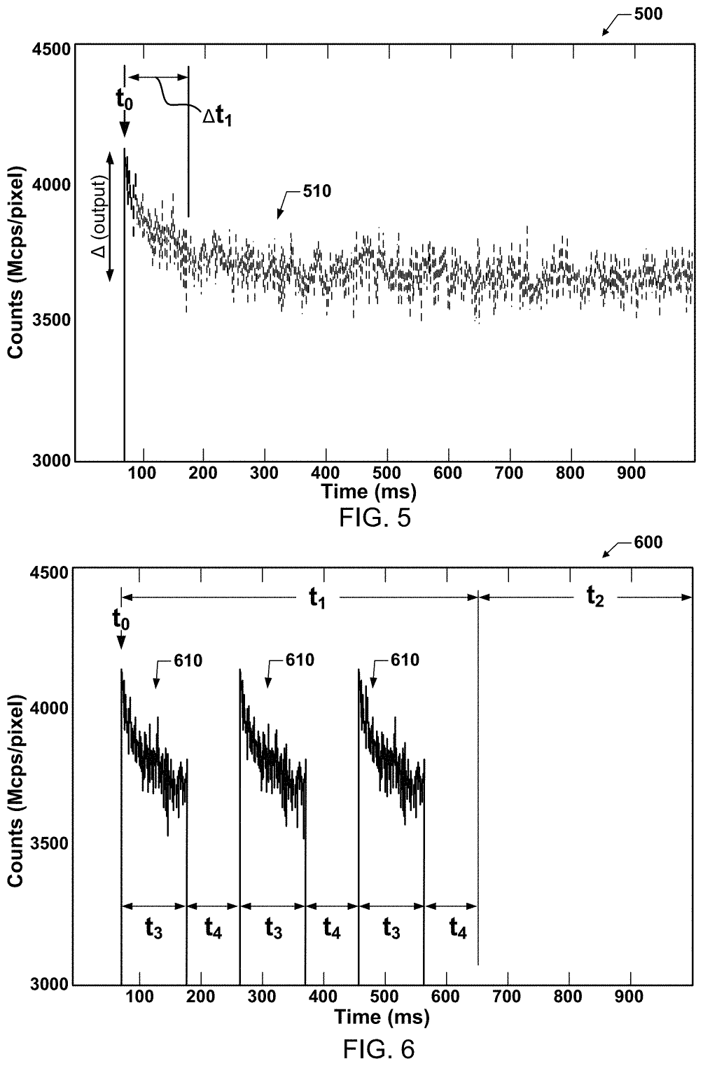

FIG. 5 illustrates is a graph 500 of the signal output from a single pixel within an X-ray detector showing the temporal nature of radiation detector output (i.e., million counts per second per detector pixel) following start of exposure to X-rays at time t₀. The graph 500 spans a one-second interval. The graph 500 shows how the count rate 510 in a CdZnTe detector pixel under a constant X-ray irradiation declines with time due to charge fields building up due to deep traps and slow hole migration. As can be seen in the graph 500, the count rate when X-ray radiation first begins exceeds the count rate at steady-state reached about 200 ms later. The initial change in output (i.e., A (output)) reflects a signal distortion of approximately 500 million counts per second (Mcps) per pixel that highlights the maximum signal distortion under conditions in which the given irradiation during the time interval Δt₁ that starts from time t₀ when the X-ray detector pixel is first exposed to X-rays. The signal distortion reflected in the drop off of the count rate is the result of the dynamic changes of the interior electric field that occurs until all the traps are filled and the density of migrating holes reach a steady state, reaching a plateau after the time interval Δt₁. The amount of signal distortion and the time required to reach the plateau are proportional to the type of the trap (life time/residence time), the energy of the trap and the density of the trap. These dynamic time-dependent output changes during signal acquisition (e.g., during a CT scan) can induce artifacts in the reconstructed images.

Various embodiments ameliorate problems caused by the temporal response of radiation detectors by limiting the time that the detectors are exposed to X-rays in a single period or pulse (i.e., t₃) and shielding the radiation detector from X-rays long enough to clear out the traps to and migrating holes (i.e., t₄) before the next exposure to X-rays. This is illustrated in graph 600 in FIG. 6, which is a plot of the signal output 610 of a single pixel over time spanning one second within an X-ray detector that includes and uses the chopper, in accordance with various embodiments. As illustrated, the chopper 150 limits each irradiation time t₃ to a period when the radiation detector is most efficient in terms of recording counts per second, and provides sufficient time in between (i.e., t₄) so that the next pulse of X-rays are detected efficiently. Specifically, the chopper may limit each irradiation time t₃ to a duration shorter than an onset time of dynamic polarization and E-field relaxation. As an example, the X-ray exposure period t₃ (described above with regard to FIGS. 4A and 4B) is illustrated as lasting approximately 100 ms, after which the count rate immediately drops to zero during the entire X-ray recovery period t₄, which is when photons from the emitted X-rays are blocked from reaching the radiation detector by the chopper. The X-ray recovery period t₄ (also described above with regard to FIGS. 4A and 4B) is illustrated as lasting approximately 90 ms. As noted above, the length of the X-ray recovery period t₄ may be chosen to ensure sufficient recovery and need not be the same or similar to the X-ray exposure period t₃. Further, the durations of data acquisition periods t₃ and detector relaxation t₄ may be modified depending upon the type of detector, individual performance of the detector, operating temperature, etc. Further, various embodiments enable dynamic adjustments to the durations of data acquisition periods t₃ and detector relaxation t₄ by varying the speed of the chopper (e.g., by controlling the speed of the drive motor 155). Thus, the system may evaluate the performance of the radiation detector (e.g., evaluating artifacts in generated images), and adjust the speed of the chopper to increase or decrease either or both of the durations of data acquisition periods t₃ and detector relaxation t₄.

The benefit of various embodiments of providing higher count rates and achieving improved dynamic response and stability by radiation detector pixels within an X-ray detector may be seen in the graph 600 in FIG. 6. Specifically, the graph 600 shows significant improvement in providing higher count rates and avoiding the plateau reached when all the traps are filled.

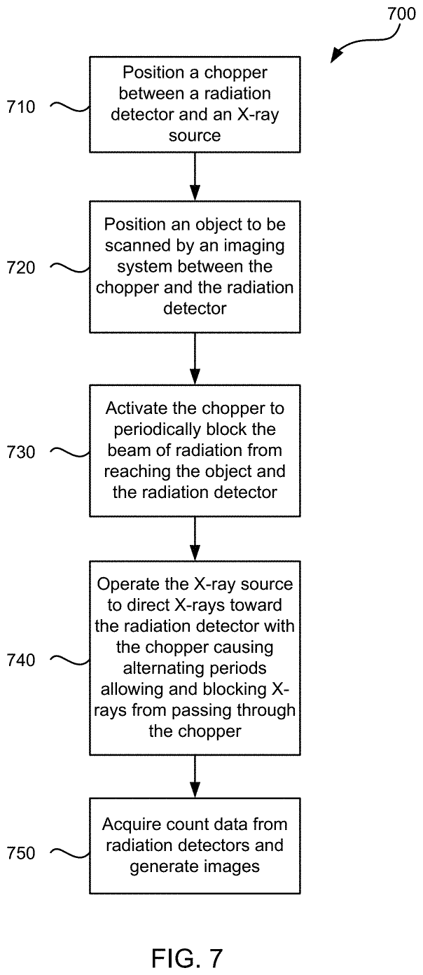

FIG. 7 illustrates a method 700 for implementing various embodiments. The method may include positioning, in an imaging system, a chopper (e.g., 150) disposed between a radiation detector (e.g., 120) and an X-ray source (e.g., 110) in block 710. In block 720, the method may include positioning an object to be scanned by the imaging system between the chopper and the radiation detector. In block 730, the chopper may be activated to periodically block the beam of radiation from reaching the object and the radiation detector. In block 740, the X-ray source may be activated to direct X-rays through an object and toward the radiation detector. The chopper will periodically interrupt the X-rays, resulting in alternating periods of X-rays striking the radiation detector and periods of no X-rays striking the radiation detector. In block 750, radiation count data from each pixel in the radiation detector may be acquired and an image generated from such data.

In some embodiments, operation of the chopper in block 730 may include adjusting a speed of the chopper so as to increase or decrease either or both of durations of data acquisition periods t₃ and detector relaxation t₄ limit exposure of the radiation detector to durations shorter than an onset time of dynamic polarization and E-field relaxation. The speed of the chopper may be adjusted or controlled in block 730 depending on the severity and amount of space charge formation. Since space charge formation may lead to artifacts in images, in some embodiments such adjustments to the speed of the chopper may be made by a computing device (e.g., 160) within the imaging system (e.g., 100) in response to detecting artifacts in images generated in block 750. In an embodiment, the detection of artifacts and the adjustments to the speed of the chopper may be made during an imaging session to improve the quality of generated images.

Various embodiments may be implemented in imaging systems used for medical imaging, such as in High-Flux applications as in X-ray Computed Tomography (CT) for medical applications, and for non-medical imaging applications, such as in baggage security scanning and industrial inspection applications.

Referring generally to all drawings, various embodiments of the present disclosure include imaging systems and methods that adjust the X-ray delivery rate in accordance with the E-field relaxation times (e.g., trapping and de-trapping rates) by varying the periods of X-ray exposure to the radiation detector to within the range of tens of microseconds to hundreds of milliseconds, which may be adjusted or controlled depending on the severity and amount of space charge formation.

Some embodiments of the present disclosure include an imaging device (100) comprising a radiation detector (120) configured to detect photon energy from ionizing radiation, a source of ionizing radiation (110) configured to emit a beam (135) of radiation toward the radiation detector, and a chopper (150) disposed between the radiation detector and the source of ionizing radiation, wherein the chopper is configured to periodically block the beam of radiation from reaching the radiation detector. In various embodiments of the imaging device, the chopper is configured to limit delivery of photon energy to the radiation detector to durations shorter than an onset time of dynamic polarization and E-field relaxation. In some embodiments of the imaging device, the chopper is a rotating chopper. In some embodiments of the imaging device, the chopper is a pneumatic shutter. In some embodiments of the imaging device, the chopper is formed from a material selected from the group tungsten, lead, or terbium. In some embodiments of the imaging device, the ionizing radiation is X-ray radiation. In some embodiments of the imaging device, the ionizing radiation is gamma radiation. In some embodiments of the imaging device, the source of ionizing radiation is configured to emit the beam of radiation at an adjustable delivery rate. In some embodiments of the imaging device, the chopper is integrated into the source of ionizing radiation such that the source of ionizing radiation is configured to emit radiation in a series of pulses. In some embodiments of the imaging device, the radiation detector comprises cadmium zinc telluride (CdZnTe). In some embodiments of the imaging device, the radiation detector is configured to alternate between on-periods of data acquisition of detected photon energy and off-periods in which no data is acquired from the radiation detector. Some embodiments of the imaging device further comprise a bias power supply (130) configured to selectively apply a voltage to the radiation detector and alternate between on-periods in which an operating voltage is applied to the radiation detector and off-periods in which no voltage is applied to the radiation detector.

Some embodiments of the present disclosure include a method of imaging an object (10) using ionizing radiation, comprising positioning a chopper (150) between a source of ionizing radiation (110) configured to emit a beam of radiation (135) and a radiation detector (120) configured to detect photon energy from ionizing radiation, positioning the object between the chopper and the radiation detector, and activating the chopper to periodically block the beam of radiation from reaching the radiation detector while the source of ionizing radiation is activated. Some embodiment methods further comprise acquiring data from the radiation detector by a computing device (160, 170), generating an image by the computing device (160) based on the acquired data, determining by the computing device whether the generated image includes an artifact, and adjusting a speed of the chopper by the computing device in response to determining that the generated image includes an artifact.

Some embodiments of the present disclosure include a chopper (150) for use in an imaging system (100), comprising a radiation-absorbing material (204, 212, 214) configured to periodically block radiation from a radiation source and periodically permit radiation from the radiation source to pass. In some embodiments the chopper comprises a rotatable disk (204) of the radiation-absorbing material configured with a plurality of spaced apart openings (206), and a drive motor (155) coupled to the rotatable disk of the radiation-absorbing material and configured to rotate the disk during operation of the imaging system. In some embodiments the chopper comprises a plurality of spaced apart blades (212) of the radiation-absorbing material coupled to a hub (202), and a drive motor (155) coupled to the hub and configured to rotate the hub during operation of the imaging system. In some embodiments the chopper comprises a plurality of spaced apart shield blocks (214) of the radiation-absorbing material coupled to a hub (202), and a drive motor (155) coupled to the hub and configured to rotate the hub during operation of the imaging system. In some embodiments the chopper comprises a shutter, such as a high-speed pneumatic shutter.

Various embodiments of the present disclosure improve the operation of imaging system using solid state radiation detectors, such as cadmium zinc telluride (CdZnTe) detectors, by suppressing temporal dynamic charge build up and polarization, which otherwise occurs during contemporary X-ray sensor operation. This provides improved dynamic responses and stability for high flux photon counting applications.

While the disclosure has been described in terms of specific embodiments, it is evident in view of the foregoing description that numerous alternatives, modifications and variations will be apparent to those skilled in the art. Each of the embodiments described herein may be implemented individually or in combination with any other embodiment unless expressly stated otherwise or clearly incompatible. Accordingly, the disclosure is intended to encompass all such alternatives, modifications and variations which fall within the scope and spirit of the disclosure and the following claims.

1. An imaging device, comprising:
a radiation detector configured to detect photon energy from ionizing radiation wherein the radiation detector is configured to alternate between on-periods of data acquisition of detected photon energy and off-periods in which no data is acquired from the radiation detector;
a source of ionizing radiation configured to emit a beam of radiation toward the radiation detector; and
a chopper disposed between the radiation detector and the source of ionizing radiation, wherein the chopper is configured to control alternating periods of exposure and periods of no exposure of material in the radiation detector to the beam of radiation, wherein the on-periods of data acquisition include at least one period of exposure and at least one period of no exposure.

2. The imaging device of claim 1, wherein the chopper is configured to limit each period of exposure to durations shorter than an onset time of dynamic polarization and E-field relaxation in the material in the radiation detector from the beam of radiation.

3. The imaging device of claim 1, wherein the chopper is a rotating chopper.

4. The imaging device of claim 1, wherein the chopper is a pneumatic shutter.

5. The imaging device of claim 1, wherein the chopper is formed from a material selected from the group tungsten, lead, or terbium.

6. The imaging device of claim 1, wherein the ionizing radiation is X-ray radiation.

7. The imaging device of claim 1, wherein the ionizing radiation is gamma radiation.

8. The imaging device of claim 1, wherein the source of ionizing radiation is configured to emit the beam of radiation at an adjustable delivery rate.

9. The imaging device of claim 1, wherein the chopper is integrated into the source of ionizing radiation such that the source of ionizing radiation is configured to emit radiation in a series of pulses.

10. The imaging device of claim 1, wherein the radiation detector comprises cadmium zinc telluride (CdZnTe).

11. (canceled)

12. The imaging device of claim 1, further comprising a bias power supply configured to selectively apply a voltage to the radiation detector and alternate between on-periods in which an operating voltage is applied to the radiation detector and off-periods in which no voltage is applied to the radiation detector.

13. A method of imaging an object using ionizing radiation, comprising:
positioning a chopper between a source of ionizing radiation configured to emit a beam of radiation and a radiation detector configured to detect photon energy from ionizing radiation;
positioning the object between the chopper and the radiation detector;
activating the chopper to control alternating periods of exposure and periods of no exposure of material in the radiation detector to the beam of radiation while the source of ionizing radiation is activated; and
operating the radiation detector to alternate between on-periods of data acquisition of detected photon energy and off-periods in which no data is acquired from the radiation detector, wherein the on-periods of data acquisition include at least one period of exposure and at least one period of no exposure.

14. The method of claim 13, further comprising:
acquiring data from the radiation detector by a computing device;
generating an image by the computing device based on the acquired data;
determining by the computing device whether the generated image includes an artifact; and
adjusting a speed of the chopper by the computing device in response to determining that the generated image includes an artifact.

15-19. (canceled)

20. The imaging device of claim 1, wherein each period of no exposure is long enough to clear out traps and migrating holes in the material in the radiation detector before the next period of exposure.

21. The imaging device of claim 1, wherein the material in the radiation detector comprises a material of an array of pixel sensors.

22. A computed tomography system, comprising:
the imaging device of claim 1; and
a computed tomography computing device configured to receive data from the on-periods of data acquisition of detected photon energy.

23. The method of claim 13, wherein activation of the chopper limits each period of exposure to durations shorter than an onset time of dynamic polarization and E-field relaxation in the material in the radiation detector from the beam of radiation.

24. The method of claim 13, wherein activation of the chopper ensures each period of no exposure is long enough to clear out traps and migrating holes in the material in the radiation detector before the next period of exposure.

25. The method of claim 13, wherein the on-periods of data acquisition comprise a whole number of periods of exposure and a whole number of periods of no exposure.
